(19) United States
(12) Patent Application Publication
(10) Pub. No.: US 2021/0297635 A1
(43) Pub. Date: Sep. 23, 2021

(54) INFORMATION PROCESSING DEVICE, INFORMATION PROCESSING METHOD, AND COMPUTER-READABLE RECORDING MEDIUM

(71) Applicant: OLYMPUS CORPORATION, Tokyo (JP)

(72) Inventors: Kazuhito HORIUCHI, Tokyo (JP); Nobuyuki WATANABE, Yokohama-shi (JP); Yoshioki KANEKO, Tokyo (JP); Hidetoshi NISHIMURA, Tokyo (JP)

(73) Assignee: OLYMPUS CORPORATION, Tokyo (JP)

(21) Appl. No.: 17/340,691

(22) Filed: Jun. 7, 2021

Related U.S. Application Data
(63) Continuation of application No. PCT/JP2018/045370, filed on Dec. 10, 2018.

(51) Int. Cl. H04N 7/18; G06F 3/01; G06K 9/32; G10L 15/22; G06K 9/62; H04N 5/232; G02B 21/36; A61B 1/05; A61B 1/00

(52) U.S. Cl. CPC H04N 7/183; G06F 3/013; G06K 9/3233; G10L 15/22; H04N 2005/2255; H04N 5/23296; G02B 21/365; A61B 1/05; A61B 1/00041; G06K 9/6215

(57) ABSTRACT

An information processing device includes: a processor including hardware. The processor is configured to analyze, based on gaze data obtained by detecting a gaze of a user and input externally, an attention degree of the gaze of the user with respect to an observation image, allocate importance corresponding to the attention degree to speech data representing speech of the user, the speech data being input externally and being associated with a same time axis as a time axis of the gaze data, record the speech data and the importance in a storage, and set a region of interest in the observation image according to the attention degree and the importance.

### CROSS-REFERENCE TO RELATED APPLICATION

This application is a continuation of International Application No. PCT/JP2018/045370, filed on Dec. 10, 2018, the entire contents of which are incorporated herein by reference.

### BACKGROUND

### 1. Technical Field

The present disclosure relates to an information processing device, an information processing method, and a computer-readable recording medium for processing speech data and gaze data.

### 2. Related Art

There has been known a technique for, in an information processing device with which a user searches for a desired region in one or a plurality of images, detecting a gaze of the user and using, for an image search, a region of interest paid attention by the user (see, for example, U.S. Pat. No. 7,593,602 B2). With this technique, the user can input the region of interest to the information processing device using the gaze. Therefore, the user can input the region of interest in a handsfree state.

### SUMMARY

In some embodiments, an information processing device includes: a processor including hardware. The processor is configured to analyze, based on gaze data obtained by detecting a gaze of a user and input externally, an attention degree of the gaze of the user with respect to an observation image, allocate importance corresponding to the attention degree to speech data representing speech of the user, the speech data being input externally and being associated with a same time axis as a time axis of the gaze data, record the speech data and the importance in a storage, and set a region of interest in the observation image according to the attention degree and the importance.

In some embodiments, provided is an information processing method executed by an information processing device. The information processing method includes: analyzing, based on gaze data obtained by detecting a gaze of a user and input externally, an attention degree of the gaze of the user with respect to an observation image; allocating importance corresponding to the attention degree to speech data representing speech of the user, the speech data being input externally and being associated with a same time axis as a time axis of the gaze data; recording the speech data and the importance in a storage; and setting a region of interest in the observation image according to the attention degree and the importance.

In some embodiments, provided is a non-transitory computer-readable recording medium with an executable program stored thereon. The program causes an information processing device to: analyze, based on gaze data obtained by detecting a gaze of a user and input externally, an attention degree of the gaze of the user with respect to an observation image; allocate importance corresponding to the attention degree to speech data representing speech of the user, the speech data being input externally and associated with a same time axis as a time axis of the gaze data; record the speech data and the importance in a storage; and set a region of interest in the observation image according to the attention degree and the importance.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a functional configuration of an information processing system according to a first embodiment;

FIG. 2 is a flowchart indicating an overview of processing executed by an information processing device according to the first embodiment;

FIG. 3 is a diagram for schematically explaining a setting method for allocation of importance to speech data by a setting unit according to the first embodiment;

FIG. 4 is a diagram schematically illustrating an example of an image displayed by a display unit according to the first embodiment;

FIG. 5 is a diagram schematically illustrating another example of the image displayed by the display unit according to the first embodiment;

FIG. 6 is a diagram illustrating a state in which FIG. 5 is divided into regions by an image analysis;

FIG. 7 is a partially enlarged view of FIG. 5;

FIG. 8 is a diagram illustrating a state in which a similar region is highlighted in FIG. 5;

FIG. 9 is a block diagram illustrating a functional configuration of an information processing system according to a second embodiment;

FIG. 10 is a flowchart illustrating an overview of processing executed by an information processing device according to the second embodiment;

FIG. 11 is a block diagram illustrating a functional configuration of an information processing system according to a third embodiment;

FIG. 12 is a flowchart illustrating an overview of processing executed by an information processing device according to the third embodiment;

FIG. 13 is a diagram for schematically explaining a setting method in which an analyzing unit according to the third embodiment sets importance in gaze data;

FIG. 14 is a diagram schematically illustrating an example of an image displayed by a display unit according to the third embodiment;

FIG. 15 is a schematic diagram illustrating the configuration of an information processing device according to a fourth embodiment;

FIG. 16 is a schematic diagram illustrating the configuration of the information processing device according to the fourth embodiment;

FIG. 17 is a block diagram illustrating a functional configuration of the information processing device according to the fourth embodiment;

FIG. 18 is a flowchart indicating an overview of processing executed by the information processing device according to the fourth embodiment;

FIG. 19 is a diagram illustrating an example of a gaze mapping image displayed by a display unit;

FIG. 20 is a diagram illustrating another example of the gaze mapping image displayed by the display unit;

FIG. 21 is a schematic diagram illustrating the configuration of a microscope system according to a fifth embodiment;

FIG. 22 is a block diagram illustrating a functional configuration of the microscope system according to the fifth embodiment;

FIG. 23 is a flowchart indicating an overview of processing executed by the microscope system according to the fifth embodiment;

FIG. 24 is a schematic diagram illustrating the configuration of an endoscope system according to a sixth embodiment;

FIG. 25 is a block diagram illustrating a functional configuration of the endoscope system according to the sixth embodiment;

FIG. 26 is a flowchart indicating an overview of processing executed by the endoscope system according to the sixth embodiment;

FIG. 27 is a diagram schematically illustrating an example of a plurality of images corresponding to a plurality of image data recorded by an image-data recording unit;

FIG. 28 is a diagram illustrating an example of an integrated image corresponding to integrated image data generated by an image processing unit;

FIG. 29 is a diagram schematically illustrating an example of an image displayed by a display unit according to the sixth embodiment; and

FIG. 30 is a diagram illustrating a state in which similar regions are highlighted in FIG. 28.

### DETAILED DESCRIPTION

Embodiments of an information processing device, an information processing method, and a program according to the disclosure are explained below with reference to the drawings. Note that the disclosure is not limited by the embodiments. The disclosure can be applied to an information processing device, an information processing method, and a program in general for performing an information search using gaze data and speech data.

In the description of the drawings, the same or corresponding components are denoted by the same reference numerals and signs as appropriate. It should be noted that the drawings are schematic and relations among dimensions of components, ratios of the components, and the like are sometimes different from real ones. Among the drawings, portions different in relations among dimensions and ratios of the dimensions thereof are sometimes included.

### First Embodiment

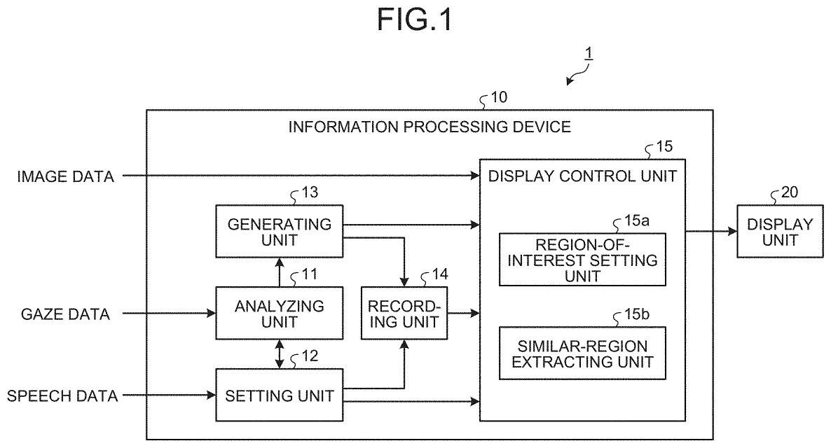

Configuration of an information processing system FIG. 1 is a block diagram illustrating a functional configuration of an information processing system according to a first embodiment. An information processing system 1 illustrated in FIG. 1 includes an information processing device 10 that performs various kinds of processing on gaze data, speech data, and image data input externally and a display unit 20 that displays various data output from the information processing device 10. Note that the information processing device 10 and the display unit 20 are connected bidirectionally by radio or wire.

Configuration of the information processing device First, the configuration of the information processing device 10 is explained.

The information processing device 10 illustrated in FIG. 1 is realized using a program installed in, for example, a server or a personal computer. Various data are input to the information processing device 10 through a network or various data acquired by an external device are input to the information processing device 10. As illustrated in FIG. 1, the information processing device 10 includes an analyzing unit 11, a setting unit 12, a generating unit 13, a recording unit 14, and a display control unit 15.

The analyzing unit 11 analyzes an attention degree of a gaze of a user on an observation image based on gaze data in a predetermined time obtained by detecting a gaze of the user and input externally. The gaze data is based on a Pupil Centre Corneal Reflection (PCCR). Specifically, the gaze data is data generated by, when a cornea of a user is irradiated with a near infrared ray from an LED light source or the like provided in a gaze detecting unit (eye tracking) not illustrated in the figure, an optical sensor, which is a gaze detecting unit, capturing an image of a pupil point and a reflection point on the cornea. The gaze data is data obtained by calculating a gaze of the user from patterns of the pupil point and the reflection point of the user based on an analysis result analyzed by performing image processing or the like on the data generated by the optical sensor capturing the images of the pupil point and the reflection point on the cornea.

Although not illustrated, when a device included in the gaze detecting unit measures gaze data, image data (an observation image) corresponding to the gaze data is presented to the user and, then, the gaze data is measured. In this case, when an image displayed to the user is fixed, that is, when an absolute coordinate does not change with time of a display area, the device including the gaze detecting unit not illustrated in the figure only has to give a relative positional relation between a measurement region and the absolute coordinate of the image to the gaze as a fixed value. The absolute coordinate indicates a coordinate represented based on predetermined one point of the image.

When a form of use is an endoscope system or an optical microscope, a field of view presented to detect a gaze is a gaze of image data. Therefore, a relative positional relation of an observation field of view with respect to an absolute coordinate of an image does not change. When the form of use is the endoscope system or the optical microscope and a moving image is recorded, in order to generate mapping data of the field of view, gaze detection data and an image recorded or an image presented simultaneously with detection of the gaze are used.

On the other hand, when a form of use is Whole Slide Imaging (WSI), a user observes a part of a slide sample of a microscope as a field of view. An observation field of view changes with time. In this case, information concerning which part of entire image data is presented as the field of view, that is, time information of switching of an absolute coordinate of a display area with respect to the entire image data is also synchronized and recorded in the same manner as information concerning a gaze and speech.

The analyzing unit 11 detects, based on gaze data in a predetermined time obtained by detecting a gaze of the user and input externally, at least one of moving speed of the gaze, a moving distance of the gaze in a fixed time, and a stagnate time of the gaze in a fixed region to thereby analyze an attention degree of the gaze (a gaze point). Note that the gaze detecting unit not illustrated in the figure may detect the gaze by capturing an image of the user by being placed in a predetermined place or may detect the gaze by capturing an image of the user by being worn by the user. Besides, the gaze data may be generated by well-known pattern matching. The analyzing unit 11 is configured using, for example, a Central Processing Unit (CPU), a Field Programmable Gate Array (FPGA), and a Graphics Processing Unit (GPU).

The setting unit 12 allocates importance corresponding to an attention degree at every predetermined time interval to speech data representing speech of the user, the speech data being input externally and being associated with the same time axis as a time axis of the gaze data, and records the speech data and the importance in the recording unit 14. Specifically, the setting unit 12 allocates, for each frame of the speech data, importance (for example, a numerical value) corresponding to the attention degree analyzed by the analyzing unit 11 at the same timing of the frame, correlates the speech data and the importance, and records the speech data and the importance in the recording unit 14. The setting unit 12 allocates high importance to the speech data immediately after the attention degree increases. The speech data representing the speech of the user input externally is generated by a speech input unit such as a microphone not illustrated in the figure at the same timing as the gaze data. The setting unit 12 is configured using a CPU, an FPGA, a GPU, and the like.

The generating unit 13 generates, on an image corresponding to image data input externally, gaze mapping data correlated with the attention degree analyzed by the analyzing unit 11 and outputs the generated gaze mapping data to the recording unit 14 and a region-of-interest setting unit 15a. Specifically, the generating unit 13 generates, for each predetermined region on the image corresponding to the image data input externally, gaze mapping data in which the attention degree analyzed by the analyzing unit 11 is correlated with coordinate information on the image. Further, the generating unit 13 correlates, in addition to the attention degree, a locus of the gaze of the user analyzed by the analyzing unit 11 on the image corresponding to the image data input externally and generates the gaze mapping data. The generating unit 13 is configured using a CPU, an FPGA, a GPU, and the like. When being used in the WSI explained above, when obtaining the gaze mapping data as the absolute coordinate of the image as explained above, the generating unit 13 uses a relative positional relation between display at the time when the gaze is measured and the absolute coordinate of the image. As explained above, when the observation field of view changes at every moment, the generating unit 13 inputs a change with time of the absolute coordinate of the display area=the field of view (for example, where in original image data the upper left of a display image is located as the absolute coordinate).

The recording unit 14 records the speech data input from the setting unit 12, the importance allocated at every predetermined time interval, and the attention degree analyzed by the analyzing unit 11 in association with one another. The recording unit 14 records the gaze mapping data input from the generating unit 13. The recording unit 14 records various programs to be executed and data being processed by the information processing device 10. The recording unit 14 is configured using a storage such as a volatile memory, a nonvolatile memory, and a recording medium.

The display control unit 15 includes the region-of-interest setting unit 15a and a similar-region extracting unit 15b. The display control unit 15 is configured using a CPU, an FPGA, a GPU, and the like. Note that the analyzing unit 11, the setting unit 12, the generating unit 13, and the display control unit 15 explained above may be configured to be able to exert the functions using at least one of the CPU, the FPGA, and the GPU or, naturally, may be configured to be able to exert the functions by combining the CPU, the FPGA, and the GPU.

The region-of-interest setting unit 15a sets a region of interest in the observation image according to the attention degree analyzed by the analyzing unit 11 and the importance input from the setting unit 12. Specifically, the region-of-interest setting unit 15a sets, as the region of interest, a region where the attention degree and the importance are equal to or larger than thresholds.

The similar-region extracting unit 15b extracts a similar region similar to the region of interest in the observation image. Specifically, the similar-region extracting unit 15b calculates feature data based on tissue characteristic such as a tint and a shape of the region of interest and extracts, from the entire observation image, as the similar region, a region where a difference from feature data of the region of interest is equal to or smaller than a predetermined threshold. The similar-region extracting unit 15b may extract, with machine learning using a convolutional neural network (CNN), a region similar to the region of interest from the observation image as the similar region.

The display control unit 15 outputs, to the display unit 20 on the outside, a gaze mapping image superimposed on gaze mapping data generated by the generating unit 13 to thereby cause the display unit 20 to display the gaze mapping image on an image corresponding to image data input externally. The display control unit 15 causes the display unit 20 to display an image highlighting the region of interest and the similar region in the gaze mapping image.

Configuration of the Display Unit

The configuration of the display unit 20 is explained.

The display unit 20 displays an image corresponding to the image data input from the display control unit 15 and gaze mapping information corresponding to the gaze mapping data input from the display control unit 15. The display unit 20 is configured using, for example, an organic Electro Luminescence (EL) or liquid crystal display monitor.

Processing of the Information Processing Device

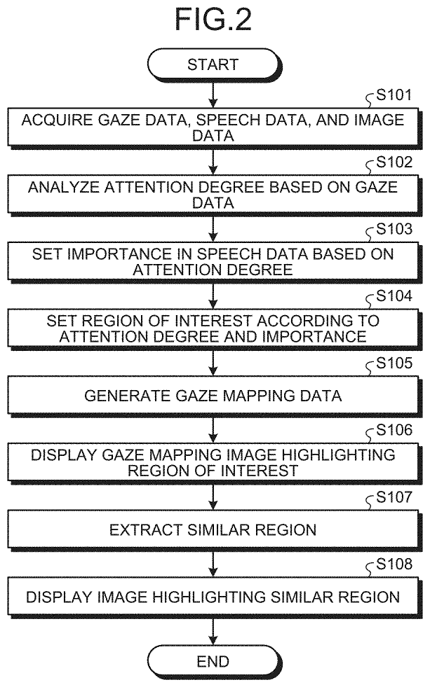

Processing of the information processing device 10 is explained. FIG. 2 is a diagram for explaining processing executed by the information processing device 10.

As illustrated in FIG. 2, first, the information processing device 10 acquires gaze data, speech data, and image data input externally (step S101).

Subsequently, the analyzing unit 11 analyzes, based on the gaze data, an attention degree of a gaze of the user with respect to an observation image (step S102). In general, it can be analyzed that, as a moving speed of the gaze is higher, the attention degree of the user is lower and, as the moving speed of the gaze is lower, the attention degree of the gaze of the user is higher. That is, the analyzing unit 11 analyzes that, as moving speed of the gaze of the user is higher, the attention degree of the gaze of the user is lower and, as the moving speed of the gaze is lower, the attention degree of the gaze of the user is higher. In this way, the analyzing unit 11 analyzes the attention degree of the gaze of the user with respect to the gaze data in every predetermined time (a time in which the user is performing observation of an image). Note that an analyzing method of the analyzing unit 11 is not limited to this. The analyzing unit 11 may analyze the attention degree of the gaze by detecting at least one of a moving distance of the gaze of the user in a fixed time and a stagnate time of the gaze of the user in a fixed region.

Thereafter, the setting unit 12 performs, on the speech data synchronized with the gaze data, setting for allocating, to the speech data, importance corresponding to the attention degree analyzed by the analyzing unit 11 at every predetermined time interval and records the importance in the recording unit 14 (step S103). After step S103, the information processing device 10 shifts to step S104 explained below.

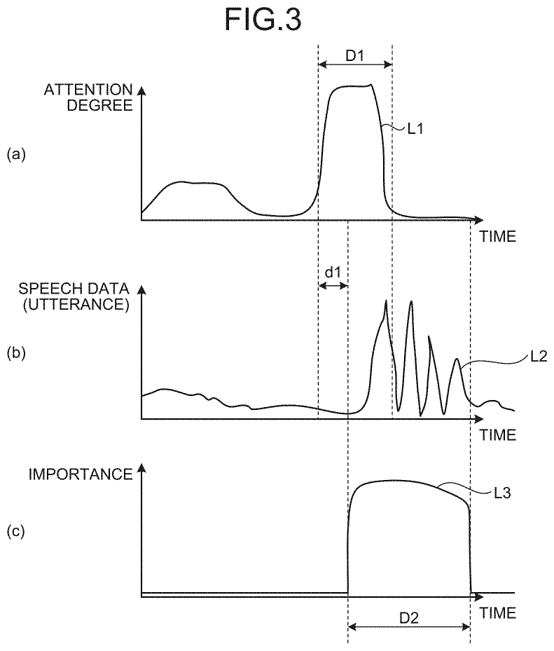

FIG. 3 is a diagram for schematically explaining a setting method for allocation of importance to speech data by the setting unit according to the first embodiment. In FIG. 3, the horizontal axis indicates time, the vertical axis of (a) of FIG. 3 indicates an attention degree, the vertical axis of (b) of FIG. 3 indicates speech data (a degree of utterance; which increases when there is utterance), and the vertical axis of (c) of FIG. 3 indicates importance. A curve L1 in (a) of FIG. 3 indicates a temporal change of the attention degree, a curve L2 in (b) of FIG. 3 indicates a temporal change of the speech data, and a curve L3 in (c) of FIG. 3 indicates a temporal change of the importance.

As indicated by the curve L1, the curve L2, and the curve L3 in FIG. 3, when the attention degree of the user is high (a section D1) and the speech data changes (a state of utterance is seen), it is highly likely that the user is uttering something important. Therefore, it can be estimated that the importance is high.

That is, the setting unit 12 performs, on the speech data, setting for allocating, to the speech data, importance corresponding to the attention degree analyzed by the analyzing unit 11 at every predetermined time interval and records the importance in the recording unit 14. Specifically, in the case illustrated in FIG. 3, the setting unit 12 performs setting for allocating high importance (for example, a numerical value, a time in which the gaze stays, or a sign indicating large, medium, or small) to the speech data in the section D1, where the analyzing unit 11 analyzes that the attention degree is high, and records the importance in the recording unit 14. At this time, when a deviation period dl occurs between the section D1, where the analyzing unit 11 analyzes that the attention degree is high, and an utterance section D2 of the speech data, the setting unit 12 performs setting for allocating high importance to the utterance section D2 immediately after the speech data corresponding to the section D1, where the analyzing unit 11 analyzes that the attention degree is high, (for example, a section after one second) and records the importance in the recording unit 14.

Note that, in the first embodiment, calibration processing for calculating a time difference between an attention degree and utterance of the user in advance (as a calibration data) and correcting deviation between the attention degree and the utterance of the user based on the calculation result may be performed.

In FIG. 3, a delay time is provided between the section D1 and the section D2 focusing on temporal deviation between the attention degree of the gaze data and the speech data. However, as a modification of FIG. 3, the setting unit 12 may provide margins before and after a section where the attention degree of the gaze data is high to thereby set a period in which the importance of the speech data is high. That is, the setting unit 12 may set a start time of the section D2 earlier than a start time of the section D1 and set an end time of the section D2 later than an end time of the section D1.

Referring back to FIG. 2, step S104 and subsequent steps are explained.

In step S104, the region-of-interest setting unit 15a sets a region of interest in the observation image according to the attention degree analyzed by the analyzing unit 11 and the importance input from the setting unit 12.

Thereafter, the generating unit 13 generates, on the image corresponding to the image data, gaze mapping data correlated with the attention degree analyzed by the analyzing unit 11 (step S105).

Subsequently, the display control unit 15 superimposes, on the image corresponding to the image data, the gaze mapping data highlighting the region of interest and outputs the gaze mapping data to the display unit 20 on the outside (step S106).

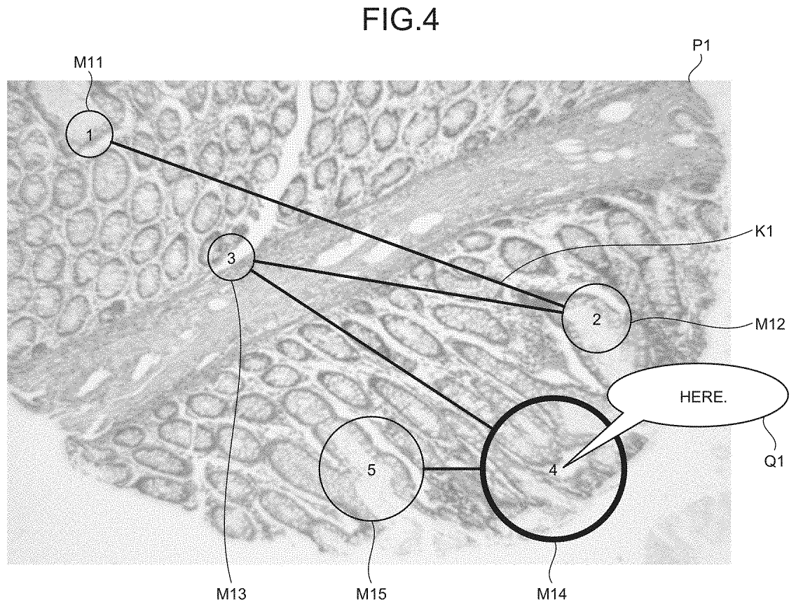

FIG. 4 is a diagram schematically illustrating an example of an image displayed by the display unit according to the first embodiment. As illustrated in FIG. 4, the display control unit 15 causes the display unit 20 to display, on an image corresponding to image data, a gaze mapping image P1 superimposed on gaze mapping data highlighting a region of interest. In FIG. 4, the display control unit 15 causes the display unit 20 to display the gaze mapping image P1 on which marks M11 to M15 of the attention degree having larger circle regions as the attention degree of the gaze is higher are superimposed. Further, the display control unit 15 causes the display unit 20 to display speech data uttered by the user in periods (times) of attention degrees near the marks M11 to M15 or to be superimposed on the marks M11 to M15 as textual information converted using a well-known character conversion technique to thereby highlight the region of interest (for example, display a frame in highlight or display the frame with a thick line). This represents that a region indicated by the mark M14 is the region of interest and the user gazes the region indicated by the mark M14 and, thereafter, utters speech “here” indicated by textual information Q1. The display control unit 15 may cause the display unit 20 to display a locus K1 of the gaze of the user and order of attention degrees as numbers.

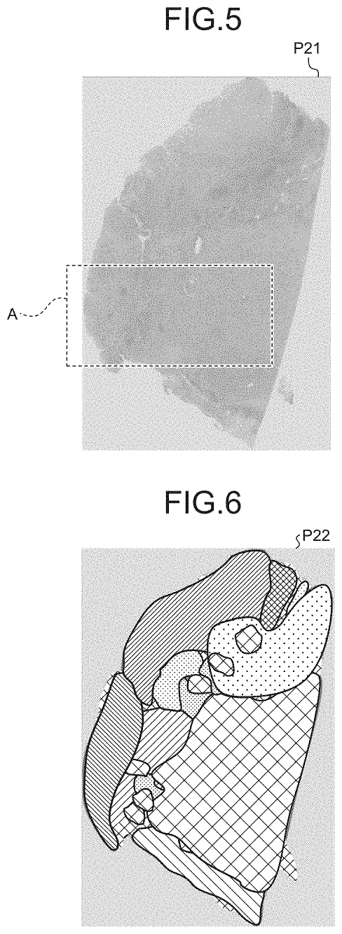

FIG. 5 is a diagram schematically illustrating another example of the image displayed by the display unit according to the first embodiment. The user observes the entire region of the observation image P21 and performs pathological diagnosis about whether or not a lesion or the like is present.

FIG. 6 is a diagram illustrating a state in which FIG. 5 is divided into regions by an image analysis. As in an image P22 illustrated in FIG. 6, according to feature data based on tissue characteristic such as tints and shapes, FIG. 5 is divided into regions having similar feature data.

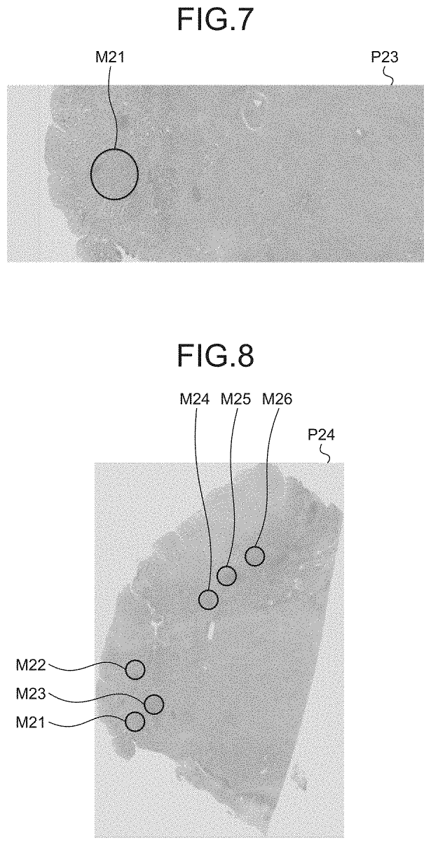

FIG. 7 is a partially enlarged view of FIG. 5. FIG. 7 corresponds to a region A in FIG. 5. The user performs observation while enlarging the observation image P21. A region M21 is set as a region of interest in an image P23 illustrated in FIG. 7.

Referring back to FIG. 2, step S107 and subsequent steps are explained.

In step S107, the similar-region extracting unit 15b extracts a similar region similar to the region of interest in the observation image. Specifically, the similar-region extracting unit 15b extracts, as the similar region, a region having feature data similar to feature data of the region of interest M21 in an image P22.

Thereafter, the display control unit 15 outputs, to the display unit 20 on the outside, an image highlighting the similar region extracted by the similar-region extracting unit 15b on the observation image P21 (step S108) After step S108, the information processing device 10 ends this processing.

FIG. 8 is a diagram illustrating a state in which the similar region is highlighted in FIG. 5. As illustrated in FIG. 8, the display control unit 15 causes the display unit 20 to display an image P24 highlighting (for example, encircling) similar regions M22 to M26, similar to the region of interest M21, extracted by the similar-region extracting unit 15b on the observation image P21.

According to the first embodiment explained above, the region-of-interest setting unit 15a sets the region of interest, which is the region paid attention by the user, based on the attention degree of the gaze and the utterance of the user. The similar-region extracting unit 15b extracts the similar region similar to the region of interest. Consequently, the user can extract a region similar to a lesion or the like that the user desires to search. As a result, it is possible to efficiently perform diagnosis and prevent a lesion from being overlooked.

In the first embodiment, the recording unit 14 records the speech data to which the importance is allocated by the setting unit 12. Therefore, it is possible to easily acquire learning data in learning a correspondence relation between image data based on mapping of a gaze and speech used in machine learning such as deep learning.

### Second Embodiment

A second embodiment of the present disclosure is explained. In the first embodiment explained above, the similar-region extracting unit 15b extracts the similar region in the observation image. However, in a second embodiment, the similar-region extracting unit 15b extracts a similar region in an image group including images stored in a database. In the following explanation, the configuration of an information processing system according to the second embodiment is explained. Thereafter, processing executed by an information processing device according to the second embodiment is explained. Note that the same components as the components of the information processing system according to the first embodiment explained above are denoted by the same reference numerals and signs. Detailed explanation of the components is omitted.

Configuration of an Information Processing System

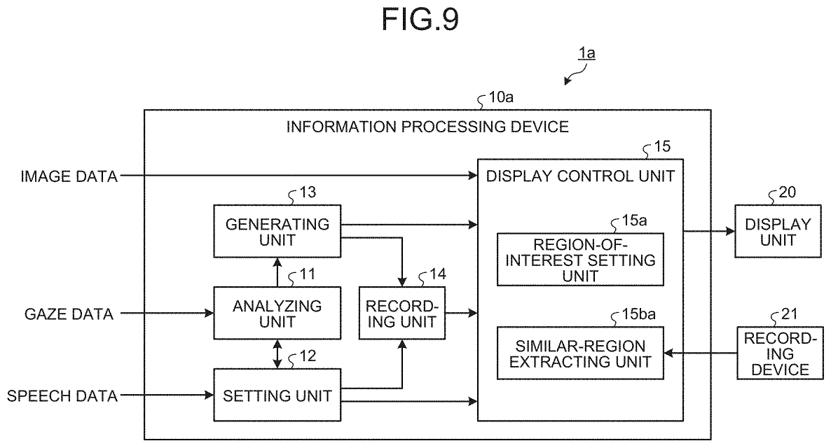

FIG. 9 is a block diagram illustrating a functional configuration of the information processing system according to the second embodiment. An information processing system 1a illustrated in FIG. 9 includes an information processing device 10a instead of the information processing device 10 according to the first embodiment explained above. The information processing device 10a includes a similar-region extracting unit 15ba instead of the similar-region extracting unit 15b according to the first embodiment explained above. The similar-region extracting unit 15ba is connected to a recording device 21.

The recording device 21 is, for example, a server connected via the Internet line. In the recording device 21, a database, in which an image group including a plurality of images is stored, is constructed.

The similar-region extracting unit 15ba extracts a region similar to the region of interest in the image group including the images stored in the database of the recording device 21.

Processing of the Information Processing Device

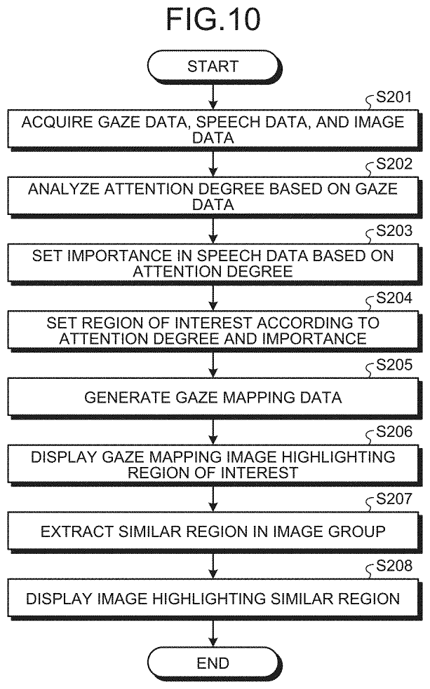

Processing executed by the information processing device 10a is explained. FIG. 10 is a flowchart indicating an overview of processing executed by the information processing device according to the second embodiment. In FIG. 10, steps S201 to S206 respectively correspond to steps S101 to S106 in FIG. 2 explained above. A user observes any one or a plurality of images recorded in the recording device 21. The region-of-interest setting unit 15a sets a region of interest based on a gaze and utterance of the user at this time.

In step S207, the similar-region extracting unit 15ba extracts a region similar to the region of interest in the image group including the images stored in the database of the recording device 21.

Subsequently, the display control unit 15 outputs, to the display unit 20 on the outside, an image highlighting the similar region extracted by the similar-region extracting unit 15ba (step S208). Specifically, the display control unit 15 highlights and displays, as a list, the similar region in each image including the similar region.

According to the second embodiment explained above, when a lesion or the like is searched from a plurality of images captured in advance, an image including a region similar to a gazed lesion part is automatically extracted. Therefore, it is possible to efficiently perform diagnosis and prevent a lesion from being overlooked.

### Third Embodiment

A third embodiment of the present disclosure is explained below. In the first embodiment explained above, the setting unit 12 allocates the importance corresponding to the attention degree analyzed by the analyzing unit 11 to the speech data and records the importance in the recording unit. However, in the third embodiment, the setting unit 12 allocates importance corresponding to a attention degree and an important word included in speech data and records the importance in the recording unit 14. In the following explanation, the configuration of the information processing system according to the third embodiment is explained. Thereafter, processing executed by an information processing device according to the third embodiment is explained. Note that the same components as the components of the information processing system according to the first embodiment explained above are denoted by the same reference numerals and signs. Detailed explanation of the components is omitted.

Configuration of an Information Processing System

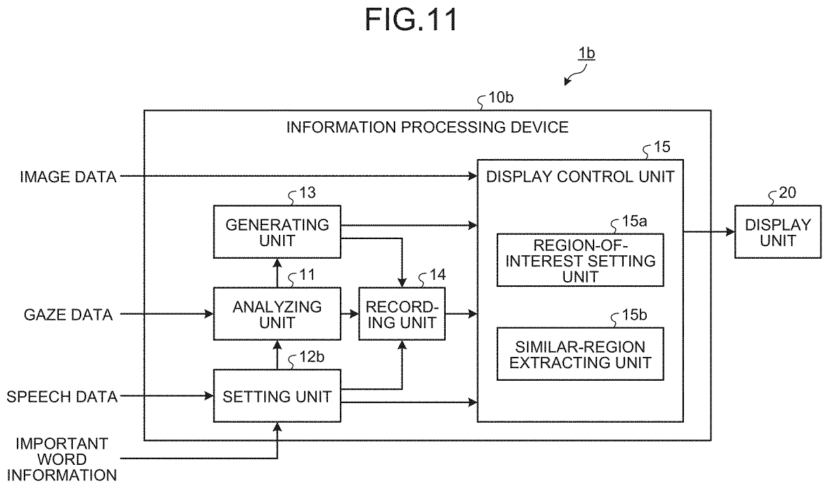

FIG. 11 is a block diagram illustrating a functional configuration of the information processing system according to the third embodiment. An information processing system 1b illustrated in FIG. 11 includes an information processing device 10b instead of the information processing device 10 according to the first embodiment explained above. The information processing device 10b includes a setting unit 12b instead of the setting unit 12 according to the first embodiment explained above.

The setting unit 12b sets an important period of speech data representing speech of a user input externally. Specifically, the setting unit 12b sets, based on important word information input externally, an important period of speech data representing speech of the user input externally. For example, when keywords input externally are cancer, bleeding, and the like and indexes of the keywords are “10”, “8”, and the like, the setting unit 12b sets, using well-known speech pattern matching or the like, as an important period, a period (a section or a time) in which the keywords are uttered. Speech data representing speech of the user input externally is generated by a speech input unit such as a microphone not illustrated in the figure. Note that the setting unit 12b may set the important period to include, for example, approximately one second to two seconds before and after the period in which the keywords are uttered. The setting unit 12b is configured using a CPU, an FPGA, a GPU, and the like. Note that, as the important word information, important word information stored in a database (speech data or textual information) in advance may be used or important word information input by the user (speech data and keyboard input) may be used.

Processing of the Information Processing Device

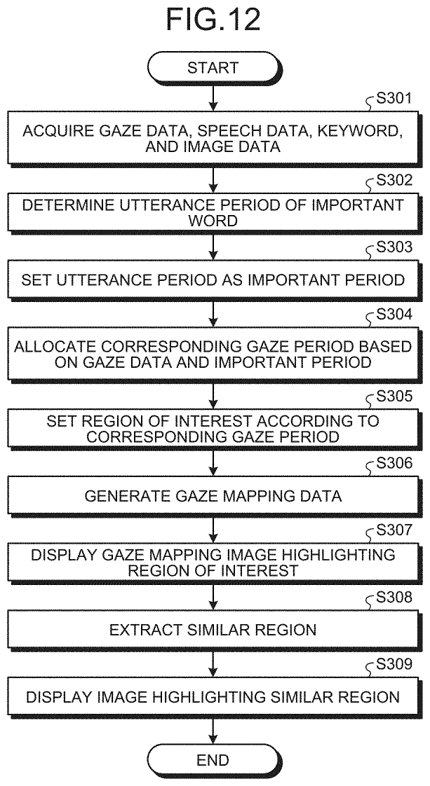

Processing executed by the information processing device 10b is explained. FIG. 12 is a flowchart indicating an overview of processing executed by the information processing device according to the third embodiment. As illustrated in FIG. 12, first, the information processing device 10b acquires gaze data, speech data, a keyword, and image data input externally (step S301).

Subsequently, the setting unit 12b determines, based on the keyword input externally, an utterance period in which the keyword, which is an important word, is uttered in the speech data (step S302) and sets, as an important period, the utterance period in which the important word is uttered in the speech data (step S303). After step S303, the information processing device 10b shifts to step S304 explained below.

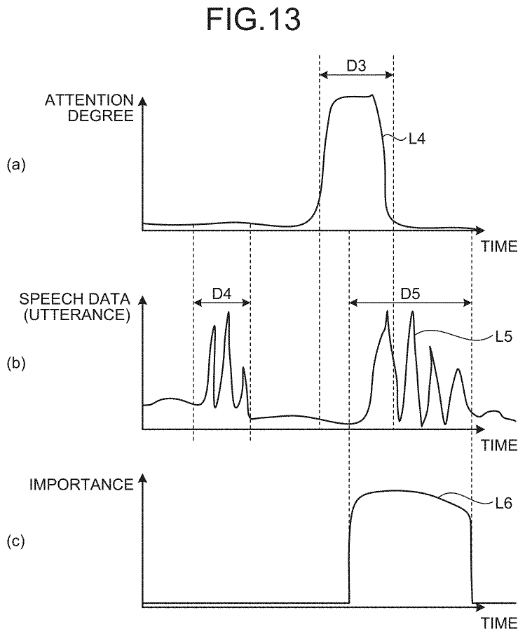

FIG. 13 is a diagram for schematically explaining a setting method in which an analyzing unit according to the third embodiment sets importance in gaze data. In FIG. 13, the horizontal axis indicates time, the vertical axis of (a) of FIG. 13 indicates an attention degree, the vertical axis of (b) of FIG. 13 indicates speech data (a degree of utterance), and the vertical axis of (c) of FIG. 13 indicates importance. A curve L4 in (a) of FIG. 13 indicates a temporal change of the attention degree, a curve L5 in (b) of FIG. 13 indicates a temporal change of the speech data, and a curve L6 in (c) of FIG. 13 indicates a temporal change of the importance.

As illustrated in (b) of FIG. 13, the setting unit 12b sets, as an important period D5, a period before and after a time (a section D3) when the attention degree of the user is high and before and after a period in which an important word is uttered. By using well-known speech pattern matching for the speech data, when a keyword of an important word input externally is “cancer”, the setting unit 12b sets, as the important period D5, in which the importance is high, a period before and after an utterance period (an utterance time) of speech data in which “cancer” is uttered. In contrast, the setting unit 12b does not set, as an important period, a period D4 in which the user utters speech but the keyword of the important word is not included. Note that, besides using the well-known speech pattern matching, the setting unit 12b may convert the speech data into textual information and, thereafter, set, with respect to the textual information, a period corresponding to the keyword as an important period in which importance is high. Even when the important word is uttered, an important period is not set when a section in which an attention degree of the user is absent before and after a period in which the important word is uttered.

Referring back to FIG. 12, step S304 and subsequent steps are explained.

In FIG. 12, in step S304, the information processing device 10b allocates, to gaze data of the user, the gaze data being associated with the same time axis as the time axis of the speech data, a gaze period corresponding to an index (for example, in the case of “cancer”, the index is “10”) allocated to a keyword of an important word in a period (a time) corresponding to the important period of the speech data set by the setting unit 12b and records the speech data and the gaze data in the recording unit 14 in synchronization with each other. After step S304, the information processing device 10b shifts to step S305 explained below.

As illustrated in FIG. 13, the analyzing unit 11 sets, based on the period D5 in which the importance of the speech set by the setting unit 12b is set, a period of gaze data corresponding to the period D5.

Note that, in the third embodiment, calibration processing for calculating a time difference between an attention degree and utterance of the user in advance (as a calibration data) and correcting shift of the attention degree and the utterance of the user based on the calculation result may be performed. A period in which a keyword having high speech importance may be simply set as an important period. A period before and after a fixed time of the important period or a period shifted from the important period may be set as a corresponding gaze period.

Referring back to FIG. 12, step S305 and subsequent steps are explained.

In step S305, the region-of-interest setting unit 15a sets a region of interest in the observation image according to the corresponding gaze period analyzed by the analyzing unit 11.

In step S306, the generating unit 13 generates gaze mapping data correlated with the corresponding gaze period analyzed by the analyzing unit 11 on the image corresponding to the image data.

Subsequently, the display control unit 15 superimposes, on the image corresponding to the image data, gaze mapping data highlighting the region of interest and outputs the gaze mapping data to the display unit 20 on the outside (step S307).

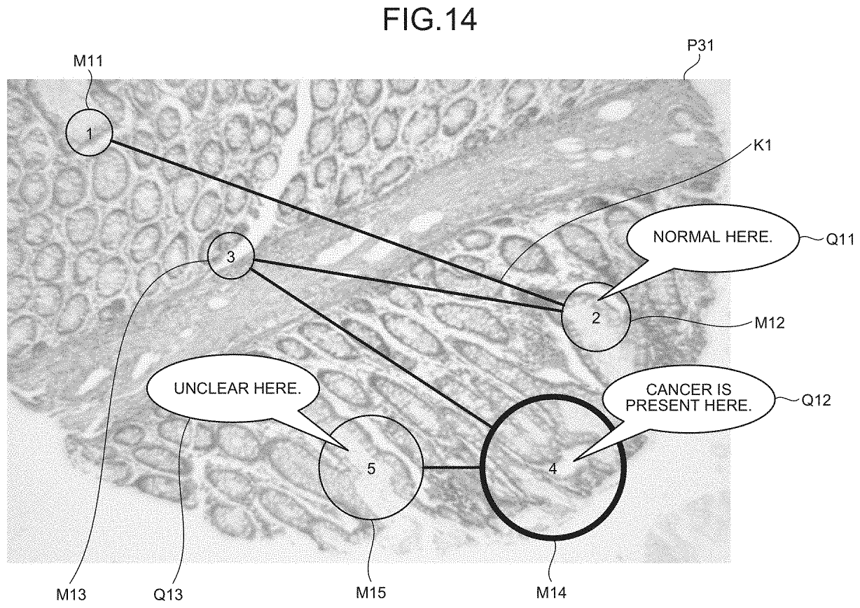

FIG. 14 is a diagram schematically illustrating an example of an image displayed by a display unit according to a third embodiment. As illustrated in FIG. 14, the display control unit 15 causes the display unit 20 to display, on an image corresponding to image data, a gaze mapping image P31 superimposed gaze mapping data highlighting a region of interest. In FIG. 14, the display control unit 15 causes the display unit 20 to display the gaze mapping image P31 on which the marks M11 to M15 of the attention degree having larger circle regions as the attention degree of the gaze is higher are superimposed. Further, the display control unit 15 may cause the display unit 20 to display textual information (for example, messages Q11 to Q13), which is obtained by converting speech data uttered by the user in periods (times) of corresponding gaze periods using a well-known character conversion technique, near the marks M11 to M15 or to be superimposed on the marks M11 to M15. The display control unit 15 causes the display unit 20 to highlight the region of interest (for example, display a frame in highlight or display the frame with a thick line). This represents that a region indicated by the mark M14 is the region of interest and the user gazes the region indicated by the mark M14 and, thereafter, utters an important word (for example, messages Q12 is included an important word “cancer”). The display control unit 15 may cause the display unit 20 to display a locus K1 of the gaze of the user and order of attention degrees as numbers.

Referring back to FIG. 12, step S308 and subsequent steps are explained.

In step S308, the similar-region extracting unit 15b extracts a similar region similar to the region of interest in the observation image (step S308).

Thereafter, the display control unit 15 outputs an image highlighting the similar region extracted by the similar-region extracting unit 15b on the observation image P21, to the display unit 20 on the outside (step S309). After step S309, the information processing device 10 ends this processing.

According to the third embodiment explained above, since the region-of-interest setting unit 15a extracts the similar region according to the important word, it is possible to extract an important region more surely. As a result, an effect of preventing the important region from being overlooked is higher.

### Fourth Embodiment

A fourth embodiment of the present disclosure is explained. In the first embodiment, each of the gaze data and the speech data is input externally. However, in the fourth embodiment, gaze data and speech data are generated. In the following explanation, the configuration of an information processing device according to the fourth embodiment is explained. Thereafter, processing executed by the information processing device according to the fourth embodiment is explained. Note that the same components as the components of the information processing system 1 according to the first embodiment explained above are denoted by the same reference numerals and signs. Detailed explanation of the components is omitted as appropriate.

Configuration of the Information Processing Device

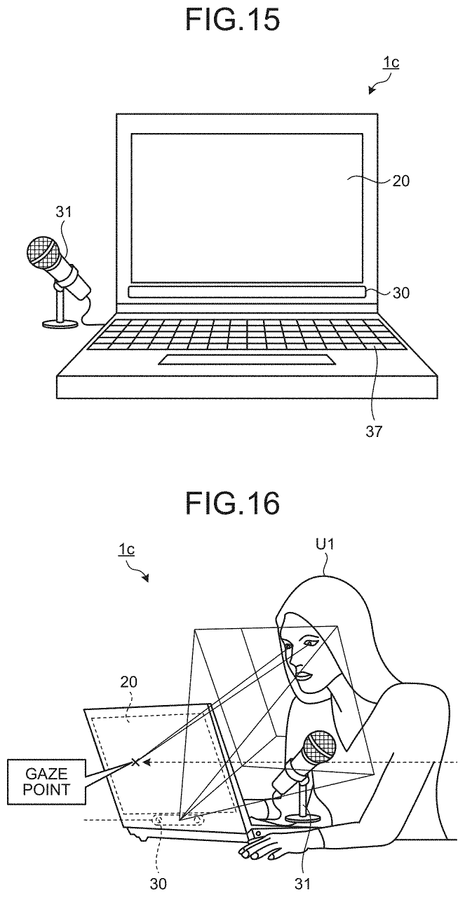

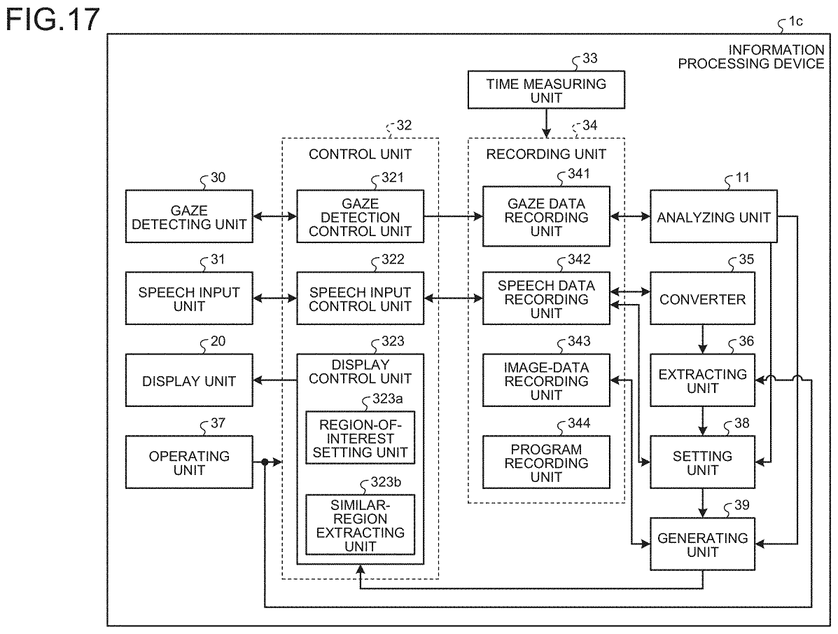

FIG. 15 is a schematic diagram illustrating the configuration of the information processing device according to the fourth embodiment. FIG. 16 is a schematic diagram illustrating the configuration of the information processing device according to the fourth embodiment. FIG. 17 is a block diagram illustrating a functional configuration of the information processing device according to the fourth embodiment.

An information processing device 1c illustrated in FIGS. 15 to 17 includes the analyzing unit 11, the display unit 20, a gaze detecting unit 30, a speech input unit 31, a control unit 32, a time measuring unit 33, a recording unit 34, a converter 35, an extracting unit 36, an operating unit 37, a setting unit 38, and a generating unit 39.

The gaze detecting unit 30 is configured using an LED light source that irradiates a near infrared ray and an optical sensor (for example, a CMOS or a CCD) that captures images of a pupil point and a reflection point on a cornea. The gaze detecting unit 30 is provided on a side surface of a casing of an information processing device 1c in which a user U1 is capable of recognizing the display unit 20 (see FIGS. 15 and 16). The gaze detecting unit 30 generates, under control by the control unit 32, gaze data obtained by detecting a gaze of the user U1 on an image displayed by the display unit 20 and outputs the gaze data to the control unit 32. Specifically, the gaze detecting unit 30 irradiates, under the control by the control unit 32, the cornea of the user U1 with a near infrared ray from the LED light source or the like. The optical sensor captures images of a pupil point and a reflection point on the cornea of the user U1 to thereby generate gaze data. The gaze detecting unit 30 continuously calculates, under the control by the control unit 32, based on an analysis result obtained by analyzing the data generated by the optical sensor with image processing or the like, gaze of the user from patterns of the pupil point and the reflection point of the user U1 to thereby generate gaze data in a predetermined time and outputs the gaze data to a gaze-detection control unit 321 explained below. Note that the gaze detecting unit 30 may simply detect a pupil of the user U1 by using well-known pattern matching with only an optical sensor to thereby generate gaze data of a detected gaze of the user U1 or may detect the gaze of the user U1 using another sensor or another well-known technique to thereby generate the gaze data.

The speech input unit 31 is configured, as a speech receiver, using a microphone to which speech is input and a speech codec that converts the speech, the input of which is received by the microphone, into digital speech data and amplifies the speech data to thereby output the speech data to the control unit 32. The speech input unit 31 receives, under the control by the control unit 32, an input of speech of the user U1 to thereby generate speech data and outputs the speech data to the control unit 32. Note that, besides the input of the speech, a speaker or the like that can output speech may be provided and a speech output function may be provided in the speech input unit 31.

The control unit 32 is configured using a CPU, an FPGA, a GPU, and the like and controls the gaze detecting unit 30, the speech input unit 31, and the display unit 20. The control unit 32 includes the gaze-detection control unit 321, a speech-input control unit 322, and a display control unit 323.

The gaze-detection control unit 321 controls the gaze detecting unit 30. Specifically, the gaze-detection control unit 321 causes the gaze detecting unit 30 to irradiates a near infrared ray to the user U1 at every predetermined timing and causes the gaze detecting unit 30 to capture an image of the pupil of the user U1 to thereby generate gaze data. The gaze-detection control unit 321 performs various kinds of image processing on the gaze data input from the gaze detecting unit 30 and outputs the gaze data to the recording unit 34.

The speech-input control unit 322 controls the speech input unit 31, performs various kinds of processing, for example, gain-up and noise reduction processing on the speech data input from the speech input unit 31, and outputs the speech data to the recording unit 34.

The display control unit 323 controls a display form of the display unit 20. The display control unit 323 includes a region-of-interest setting unit 323a and a similar-region extracting unit 323b.

The region-of-interest setting unit 323a sets a region of interest in an observation image according to an attention degree analyzed by the analyzing unit 11 and importance input from the setting unit 38.

The similar-region extracting unit 323b extracts a similar region similar to the region of interest in the observation image.

The display control unit 323 causes the display unit 20 to display an image corresponding to the image data recorded in the recording unit 34 or a gaze mapping image corresponding to the gaze mapping data generated by the generating unit 39.

The time measuring unit 33 is configured using a timer, a clock generator, and the like and gives time information to the gaze data generated by the gaze detecting unit 30, the speech data generated by the speech input unit 31, and the like.

The recording unit 34 is configured using a volatile memory, a nonvolatile memory, a recording medium, and the like and records various kinds of information concerning the information processing device 1c. The recording unit 34 includes a gaze-data recording unit 341, a speech-data recording unit 342, an image-data recording unit 343, and a program recording unit 344.

The gaze-data recording unit 341 records gaze data input from the gaze-detection control unit 321 and outputs the gaze data to the analyzing unit 11.

The speech-data recording unit 342 records speech data input from the speech-input control unit 322 and outputs the speech data to the converter 35.

The image-data recording unit 343 records a plurality of image data. The plurality of image data are data input externally of the information processing device 1c or data captured by an external imaging device by a recording medium.

The program recording unit 344 records various programs to be executed by the information processing device 1c, data (for example, dictionary information registering keywords and text conversion dictionary information) used during execution of the various programs, and processing data during the execution of the various programs.

The converter 35 performs well-known text conversion processing on the speech data to thereby convert the speech data into textual information (text data) and outputs the textual information to the extracting unit 36.

Note that a configuration for not performing speech-to-character conversion immediately after the speech data input is also possible. In that case, importance may be set for speech information. Thereafter, the speech information may be converted into textual information.

The extracting unit 36 extracts, from the textual information converted by the converter 35, characters and words (keywords) corresponding to an instruction signal input from the operating unit 37 explained below and outputs a result of the extraction to the setting unit 38. Note that, when an instruction signal is not input from the operating unit 37 explained below, the extracting unit 36 outputs the textual information input from the converter 35 to the setting unit 38.

The operating unit 37 is configured using a mouse, a keyboard, a touch panel, various switches, and the like, receives an input of operation by the user U1, and outputs content of the operation, the input of which is received, to the control unit 32.

The setting unit 38 allocates, based on an attention degree analyzed by the analyzing unit 11 at every predetermined time interval and the textual information extracted by the extracting unit 36, importance and the textual information converted by the converter 35 to speech data associated with the same time axis as the time axis of the gaze data and records the importance and the textual information in the recording unit 34.

The generating unit 39 generates, on the image corresponding to the image data displayed by the display unit 20, gaze mapping data correlated with the attention degree analyzed by the analyzing unit 11 and the textual information converted by the converter 35 and outputs the gaze mapping data to the image-data recording unit 343 and the display control unit 323.

Processing of the Information Processing Device

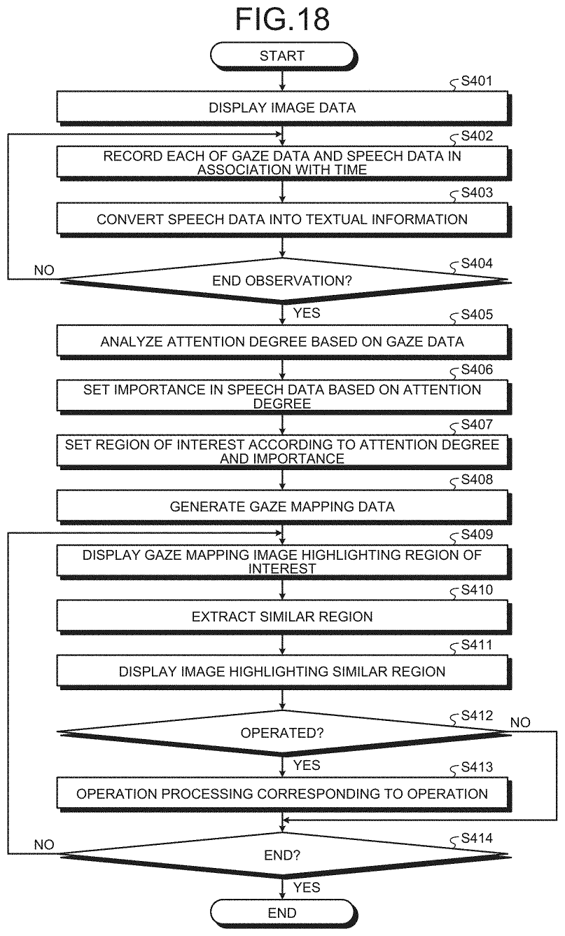

Processing executed by the information processing device 1c is explained. FIG. 18 is a flowchart illustrating an overview of processing executed by the information processing device according to the fourth embodiment.

As illustrated in FIG. 18, first, the display control unit 323 causes the display unit 20 to display an image corresponding to image data recorded by the image-data recording unit 343 (step S401). In this case, the display control unit 323 causes the display unit 20 to display an image corresponding to image data selected according to operation of the operating unit 37.

Subsequently, the control unit 32 records each of gaze data generated by the gaze detecting unit 30 and speech data generated by the speech input unit 31 and a time measured by the time measuring unit 33 in the gaze-data recording unit 341 and the speech-data recording unit 342 in association with each other (step S402).

Thereafter, the converter 35 converts the speech data recorded by the speech-data recording unit 342 into textual information (step S403). Note that this step may be performed after S406 explained below.

Subsequently, when an instruction signal for ending observation of the image displayed by the display unit 20 is input from the operating unit 37 (step S404: Yes), the information processing device 1c shifts to step S405 explained below. In contrast, when the instruction signal for ending the observation of the image displayed by the display unit 20 is not input from the operating unit 37 (step S404: No), the information processing device 1c returns to step S402.

Step S405 corresponds to step S102 in FIG. 2 explained above. After step S405, the information processing device 1c shifts to step S406 explained below.

In step S406, the setting unit 38 allocates, based on an attention degree analyzed by the analyzing unit 11 at every predetermined time interval and textual information extracted by the extracting unit 36, importance and textual information converted by the converter 35 to speech data associated with the same time axis as the time axis of the gaze data and records the importance and the textual information in the recording unit 34. In this case, the setting unit 38 performs weighting of the importance of the speech data corresponding to the textual information extracted by the extracting unit 36 and records the importance in the recording unit 34. For example, the setting unit 38 allocates, as the importance, to the speech data, a value obtained by multiplying the attention degree by a coefficient based on the textual information extracted by the extracting unit 36 and records the value in the recording unit 34.

Thereafter, the region-of-interest setting unit 323a sets a region of interest in the observation image according to the attention degree analyzed by the analyzing unit 11 and the importance set by the setting unit 38 (step S407).

Subsequently, the generating unit 39 generates, on the image corresponding to the image data displayed by the display unit 20, gaze mapping data correlated with the attention degree analyzed by the analyzing unit 11, the textual information converted by the converter 35, and the region of interest set by the region-of-interest setting unit 323a (step S408).

Subsequently, the display control unit 323 causes the display unit 20 to display a gaze mapping image corresponding to the gaze mapping data generated by the generating unit 39 (step S409).

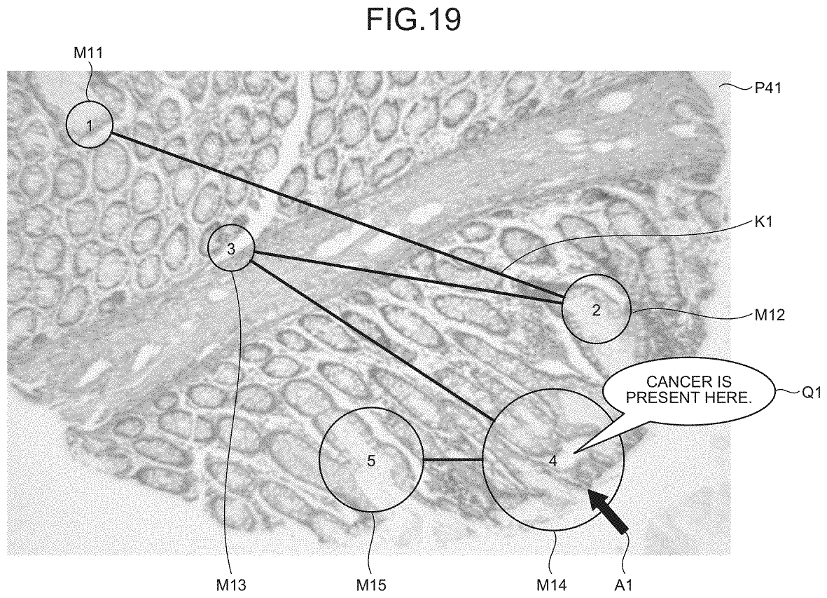

FIG. 19 is a diagram illustrating an example of a gaze mapping image displayed by the display unit. As illustrated in FIG. 19, the display control unit 323 causes the display unit 20 to display a gaze mapping image P41 corresponding to gaze mapping data generated by the generating unit 39. The marks M11 to M15 corresponding to a region of interest of the gaze and the locus K1 of the gaze are superimposed on the gaze mapping image P41. Textual information of speech data uttered at timing of the attention degree and a region of interest set by the region-of-interest setting unit 323a are correlated with the gaze mapping image P41. The numbers of the marks M11 to M15 indicate order of gaze of the user U1 and the sizes (regions) of the marks M11 to M15 indicate the magnitudes of attention degrees. Further, when the user U1 operates the operating unit 37 and moves a cursor A1 to a desired position, for example, the mark M14, the textual information Q1, for example, “cancer is present here” correlated with the mark M14 is displayed. A region of interest indicated by the mark M14 is highlighted (for example, a frame is displayed in highlight or displayed by a thick line). Note that, in FIG. 19, the display control unit 323 causes the display unit 20 to display the textual information. However, for example, the display control unit 323 may convert the textual information into speech to thereby output speech data. Consequently, the user U1 can intuitively understand important speech content and a gazing region. Further, the user U1 can intuitively understand a locus of a gaze at the time of observation by the user U1.

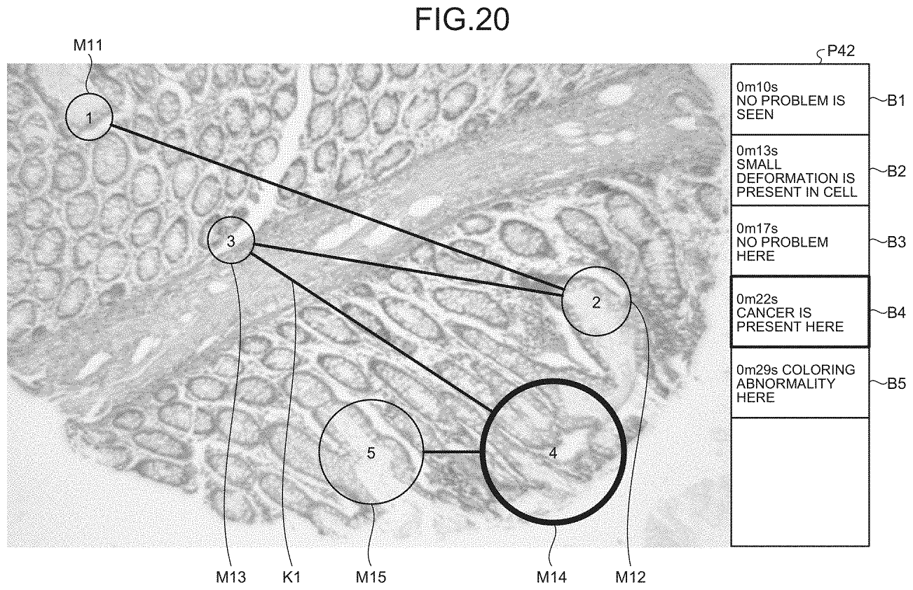

FIG. 20 is a diagram illustrating another example of the gaze mapping image displayed by the display unit. As illustrated in FIG. 20, the display control unit 323 causes the display unit 20 to display a gaze mapping image P42 corresponding to the gaze mapping data generated by the generating unit 39. Further, the display control unit 323 causes the display unit 20 to display icons B1 to B5 associated with textual information and times when the textual information is uttered. Further, the display control unit 323 causes the display unit 20 to highlight the mark M14, which is the region of interest, and causes the display unit 20 to highlight textual information, for example, the icon B4 corresponding to a time of the mark M14 (for example, display a frame in highlight or display the frame with a thick line). Consequently, the user U1 can intuitively understand important speech content and a gazing region and can intuitively understand content of utterance.

Referring back to FIG. 18, step S410 and subsequent steps are explained.

In step S410, the similar-region extracting unit 323b extracts a similar region similar to the region of interest in the observation image. Specifically, the similar-region extracting unit 323b extracts, as a similar region, a region similar to the region of interest in the image P41 or the image P42.

Thereafter, the display control unit 323 outputs, to the display unit 20 on the outside, an image highlighting the similar region extracted by the similar-region extracting unit 323b on the image P41 or the image P42 (step S411).

Subsequently, when any one of marks corresponding to a plurality of regions of interest is operated by the operating unit 37 (step S412: Yes), the control unit 32 executes operation processing corresponding to the operation (step S413). Specifically, the display control unit 323 causes the display unit 20 to highlight a similar region similar to a mark corresponding to the region of interest selected by the operating unit 37 (see, for example, FIG. 8). The speech-input control unit 322 causes the speech input unit 31 to play speech data correlated with a region having a high attention degree. After step S413, the information processing device 1c shifts to step S414 explained below.

When any one of the marks corresponding to the plurality of regions of interest is not operated by the operating unit 37 in step S412 (step S412: No), the information processing device 1c shifts to step S414 explained below.

When an instruction signal for instructing an end of observation is input from the operating unit 37 in step S414 (step S414: Yes), the information processing device 1c ends this processing. In contrast, when an instruction signal for instructing an end of observation is not input from the operating unit 37 (step S414: No), the information processing device 1c returns to step S409 explained above.

According to the fourth embodiment explained above, the region-of-interest setting unit 323a sets, based on the attention degree of the gaze and the utterance of the user, the region of interest, which is the region paid attention by the user. The similar-region extracting unit 323b extracts the similar region similar to the region of interest. Consequently, the user can extract a region similar to a lesion or the like that the user desires to search. As a result, it is possible to efficiently perform diagnosis and prevent a lesion from being overlooked.

According to the fourth embodiment, the display control unit 323 causes the display unit 20 to display the gaze mapping image corresponding to the gaze mapping data generated by the generating unit 39. Therefore, the gaze mapping image can be used for confirmation of overlook prevention of observation by the user for an image, confirmation of a technical skill such as observation by the user, educations of observation for other users, and the like, conferences, and the like.

### Fifth Embodiment

A fifth embodiment of the present disclosure is explained. In the fourth embodiment explained above, the information processing device 1c is configured alone. However, in the fifth embodiment, an information processing device is configured by being incorporated in a part of a microscope system. In the following explanation, the configuration of the microscope system according to the fifth embodiment is explained. Thereafter, processing executed by the microscope system according to the fifth embodiment is explained. Note that the same components as the components of the information processing device 1c according to the fourth embodiment explained above are denoted by the same reference numerals and signs. Detailed explanation of the components is omitted as appropriate.

Configuration of the Microscope System

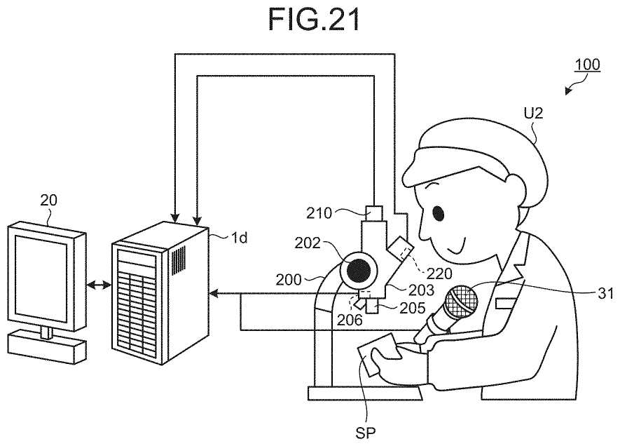

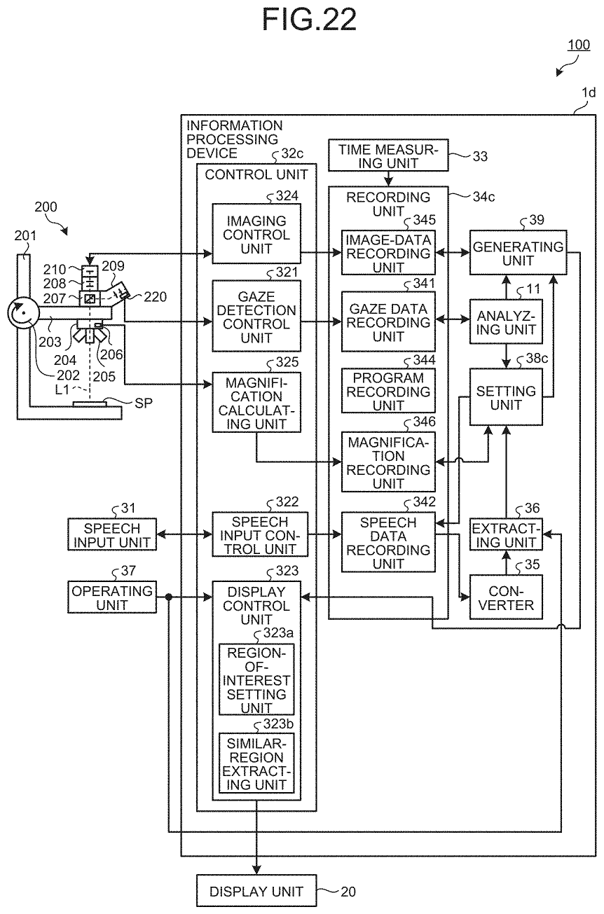

FIG. 21 is a schematic diagram illustrating the configuration of the microscope system according to the fifth embodiment. FIG. 22 is a block diagram illustrating a functional configuration of the microscope system according to the fifth embodiment.

As illustrated in FIGS. 21 and 22, a microscope system 100 includes an information processing device 1d, the display unit 20, the speech input unit 31, the operating unit 37, a microscope 200, an imaging unit 210, and a gaze detecting unit 220.

Configuration of the Microscope

First, the configuration of the microscope 200 is explained.

The microscope 200 includes a main body unit 201, a rotating unit 202, a rising and lowering unit 203, a revolver 204, an objective lens 205, a magnification detecting unit 206, a lens barrel unit 207, a connecting unit 208, and an eyepiece unit 209.

A specimen SP is placed on the main body unit 201. The main body unit 201 is formed in a substantial U-shape. The rising and lowering unit 203 is connected to the main body unit 201 using the rotating unit 202.

The rotating unit 202 rotates according to operation by a user U2 to thereby move the rising and lowering unit 203 in the vertical direction.

The rising and lowering unit 203 is provided to be movable in the vertical direction with respect to the main body unit 201. The revolver 204 is connected to a surface on one end side of the rising and lowering unit 203. The lens barrel unit 207 is connected to a surface on the other end side of the rising and lowering unit 203.

A plurality of objective lenses 205 having different magnifications one another are connected to the revolver 204. The revolver 204 is connected to the rising and lowering unit 203 to be capable of rotating with respect to an optical axis L1. The revolver 204 disposes a desired objective lens 205 on the optical axis L1 according to operation by the user U2. Note that information indicating the magnifications, for example, IC chips or labels are attached to the plurality of objective lenses 205. Note that, besides the IC chips or the labels, shapes indicating the magnifications may be provided in the objective lenses 205.

The magnification detecting unit 206 detects the magnification of the objective lens 205 disposed on the optical axis L1 and outputs a result of the detection to the information processing device 1d. The magnification detecting unit 206 is configured using means for detecting, for example, the position of the revolver 204 for object switching.

The lens barrel unit 207 transmits a part of an object image of the specimen SP formed by the objective lens 205 to the connecting unit 208 and reflects a part of the object image to the eyepiece unit 209. The lens barrel unit 207 includes, on the inside, a prism, a half mirror, and a collimate lens.

One end of the connecting unit 208 is connected to the lens barrel unit 207. The other end of the connecting unit 208 is connected to the imaging unit 210. The connecting unit 208 guides the object image of the specimen SP transmitted through the lens barrel unit 207 to the imaging unit 210. The connecting unit 208 is configured using pluralities of collimate lenses, tube lenses, and the like.

The eyepiece unit 209 guides and focuses the object image reflected by the lens barrel unit 207. The eyepiece unit 209 is configured using pluralities of collimate lenses, tube lenses, and the like.

Configuration of the Imaging Unit

The configuration of the imaging unit 210 is explained.

The imaging unit 210 receives the object image of the specimen SP formed by the connecting unit 208 to thereby generate image data and outputs the image data to the information processing device 1d. The imaging unit 210 is configured using an image sensor such as a CMOS or a CCD, an image processing engine that applies various kinds of image processing to the image data, and the like.

Configuration of the Gaze Detecting Unit

The configuration of the gaze detecting unit 220 is explained.

The gaze detecting unit 220 is provided on the inside or the outside of the eyepiece unit 209. The gaze detecting unit 220 detects a gaze of the user U2 to thereby generate gaze data and outputs the gaze data to the information processing device 1d. The gaze detecting unit 220 is configured using an LED light source that is provided on the inside of the eyepiece unit 209 and irradiates a near infrared ray and an optical sensor (for example, a CMOS or a CCD) that is provided on the inside of the eyepiece unit 209 and captures images of a pupil point and a reflection point on a cornea. The gaze detecting unit 220 irradiates, under control by the information processing device 1d, the cornea of the user U2 with a near infrared ray from the LED light source or the like. The optical sensor captures images of the pupil point and the reflection point on the cornea of the user U2 to thereby generate data. A gaze detecting unit 220 detects, under the control by the information processing device 1d, based on an analysis result obtained by analyzing, with image processing or the like, the data generated by the optical sensor, a gaze of the user from patterns of the pupil point and the reflection point of the user U2 to thereby generate gaze data and outputs the gaze data to the information processing device 1d.

Configuration of the Information Processing Device

The configuration of the information processing device 1d is explained.

The information processing device 1d includes a control unit 32c, a recording unit 34c, and a setting unit 38c instead of the control unit 32, the recording unit 34, and the setting unit 38 of the information processing device 1c according to the fourth embodiment explained above.

The control unit 32c is configured using a CPU, an FPGA, a GPU, and the like and controls the display unit 20, the speech input unit 31, the imaging unit 210, and the gaze detecting unit 220. The control unit 32c further includes an imaging control unit 324 and a magnification calculating unit 325 in addition to the gaze-detection control unit 321, the speech-input control unit 322, and the display control unit 323 of the control unit 32 in the fourth embodiment explained above.

The imaging control unit 324 controls the operation of the imaging unit 210. The imaging control unit 324 causes the imaging unit 210 to sequentially perform imaging according to a predetermined frame rate to thereby generate image data. The imaging control unit 324 applies image processing (for example, development processing) to the image data input from the imaging unit 210 and outputs the image data to the recording unit 34c.

The magnification calculating unit 325 calculates present observation magnification of the microscope 200 based on a detection result input from the magnification detecting unit 206 and outputs the calculation result to the magnification recording unit 346 explained below. For example, the magnification calculating unit 325 calculates the present observation magnification of the microscope 200 based on magnification of the objective lens 205 input from the magnification detecting unit 206 and magnification of the eyepiece unit 209.

The recording unit 34c is configured using a volatile memory, a nonvolatile memory, a recording medium, and the like. The recording unit 34c includes an image-data recording unit 345 instead of the image-data recording unit 343 according to the fourth embodiment explained above. The image-data recording unit 345 records the image data input from the imaging control unit 324 and outputs the image data to the generating unit 39. The recording unit 34c also includes a magnification recording unit 346. The magnification recording unit 346 records the magnification data input from the magnification calculation unit 325 and outputs the magnification data to the setting unit 38c.

The setting unit 38c allocates, based on the attention degree analyzed by the analyzing unit 11 at every predetermined time interval and the calculation result of the magnification recording unit 346, importance and textual information converted by the converter 35 to speech data associated with the same time axis as the time axis of gaze data and records the importance and the textual information in the recording unit 34c. Specifically, the setting unit 38c allocates, as importance (for example, a numerical value) for each frame of the speech data, a value obtained by multiplying the attention degree analyzed by the analyzing unit 11 by a coefficient based on the calculation result of the magnification recording unit 346 and records the value in the recording unit 34c. That is, the setting unit 38c performs processing for setting importance higher as display magnification is larger. The setting unit 38c is configured using a CPU, an FPGA, a GPU, and the like.

Processing of the Microscope System

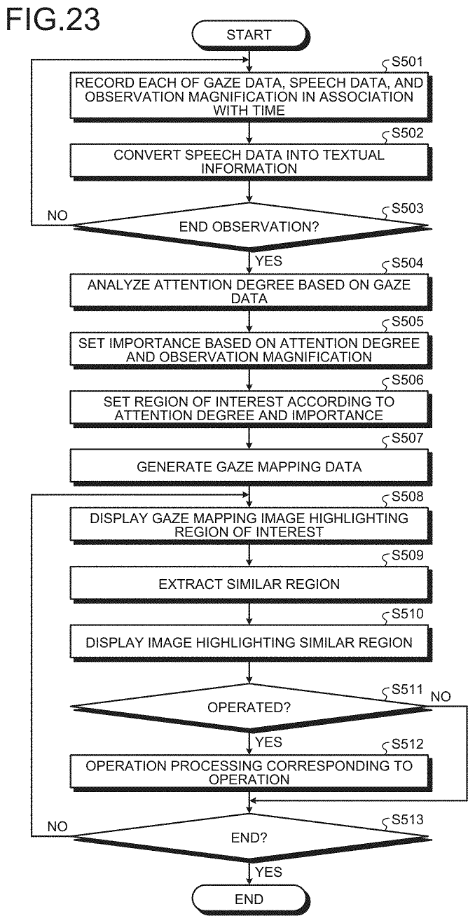

Processing executed by the microscope system 100 is explained. FIG. 23 is a flowchart indicating an overview of processing executed by the microscope system according to the fifth embodiment.

As illustrated in FIG. 23, first, the control unit 32c records each of gaze data generated by the gaze detecting unit 220, speech data generated by the speech input unit 31, and observation magnification calculated by the magnification calculating unit 325 in the gaze-data recording unit 341, the speech-data recording unit 342, and the magnification recording unit 346 in association with a time measured by the time measuring unit 33 (step S501). After step S501, the microscope system 100 shifts to step S502 explained below.

Steps S502 to S504 respectively correspond to steps S403 to S405 in FIG. 18 explained above. After step S504, the microscope system 100 shifts to step S505.

In step S505, the setting unit 38c allocates, based on an attention degree analyzed by the analyzing unit 11 at every predetermined time interval and a calculation result recorded in the magnification recording unit 346, importance and textual information converted by the converter 35 to speech data associate with the same time axis as the time axis of the gaze data and records the importance and the textual information in the recording unit 34c. After step S505, the microscope system 100 shifts to step S506.

Steps S506 to S513 respectively correspond to steps S407 to S414 in FIG. 18 explained above.

According to the fifth embodiment explained above, the importance based on the observation magnification and the attention degree are allocated to the speech data. Therefore, it is possible to set a region of interest taking into account observation content and the attention degree and efficiently observe a similar region similar to the region of interest and prevent a lesion or the like from being overlooked.

Note that, in the fifth embodiment, the observation magnification calculated by the magnification calculating unit 325 is recorded in the magnification recording unit 346. However, an operation history of the user U2 may be recorded and the importance of the speech data may be allocated further taking into account the operation history.

### Sixth Embodiment

A sixth embodiment of the present disclosure is explained. In the sixth embodiment, an information processing device is configured by being incorporated in a part of an endoscope system. In the following explanation, the configuration of the endoscope system according to the sixth embodiment is explained. Thereafter, processing executed by the endoscope system according to the sixth embodiment is explained. Note that the same components as the components of the information processing device 1c according to the fourth embodiment explained above are denoted by the same reference numerals and signs. Detailed explanation of the components is omitted as appropriate.

Configuration of the Endoscope System

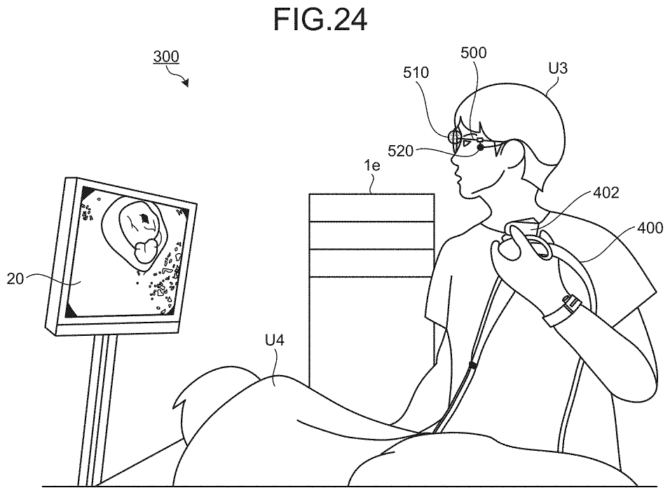

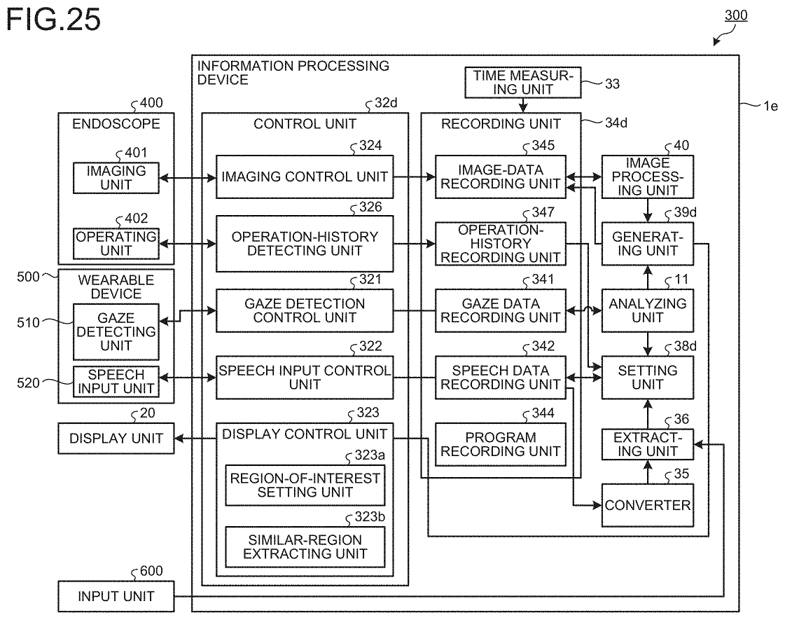

FIG. 24 is a schematic diagram illustrating the configuration of the endoscope system according to the sixth embodiment. FIG. 25 is a block diagram illustrating a functional configuration of the endoscope system according to the sixth embodiment.

An endoscope system 300 illustrated in FIGS. 24 and 25 includes the display unit 20, an endoscope 400, a wearable device 500, an input unit 600, and an information processing device 1e.

Configuration of the Endoscope

First, the configuration of the endoscope 400 is explained.

By being inserted into a subject U4 by a user U3 such as a doctor or a surgeon, the endoscope 400 captures an image of the inside of the subject U4 to thereby generate image data and outputs the image data to the information processing device 1e. The endoscope 400 includes an imaging unit 401 and an operating unit 402.

The imaging unit 401 is provided at a distal end portion of an insertion portion of the endoscope 400. The imaging unit 401 captures an image of the inside of the subject U4 under control by the information processing device 1e to thereby generate image data and outputs the image data to the information processing device 1e. The imaging unit 401 is configured using an optical system that can change observation magnification, an image sensor such as a CMOS or a CCD that receives an object image formed by the optical system to thereby generate image data, and the like.

The operating unit 402 serves as an operation receiver. The operating unit 402 receives inputs of various kinds of operation by the user U3 and outputs operation signals corresponding to the received various kinds of operation to the information processing device 1e.

Configuration of the Wearable Device

The configuration of the wearable device 500 is explained.

The wearable device 500 is attached to the user U3 and detects a gaze of the user U3 and receives an input of speech of the user U3. The wearable device 500 includes a gaze detecting unit 510 and a speech input unit 520.

The gaze detecting unit 510 is provided in the wearable device 500 and detects an attention degree of the gaze of the user U3 to thereby generate gaze data and outputs the gaze data to the information processing device 1e. The gaze detecting unit 510 has the same configuration as the configuration of the gaze detecting unit 220 according to the fifth embodiment explained above. Therefore, detailed explanation of the configuration is omitted.

The speech input unit 520 is provided in the wearable device 500 and receives an input of speech of the user U3 to thereby generate speech data and outputs the speech data to the information processing device 1e. The speech input unit 520 is configured using a microphone and the like.

Configuration of the Input Unit

The configuration of the input unit 600 is explained.

The input unit 600 is configured using a mouse, a keyboard, a touch panel, and various switches. The input unit 600 receives inputs of various kinds of operation by the user U3 and outputs operation signals corresponding to the received various kinds of operation to the information processing device 1e.

Configuration of the Information Processing Device

The configuration of the information processing device le is explained.

The information processing device 1e includes a control unit 32d, a recording unit 34d, a setting unit 38d, and a generating unit 39d instead of the control unit 32c, the recording unit 34c, the setting unit 38c, and the generating unit 39 of the information processing device 1d according to the fifth embodiment explained above. Further, the information processing device 1d includes an image processing unit 40.

The control unit 32d is configured using a CPU, an FPGA, a GPU, and the like and controls the endoscope 400, the wearable device 500, and the display unit 20. The control unit 32d includes an operation-history detecting unit 326 in addition to the gaze-detection control unit 321, the speech-input control unit 322, the display control unit 323, and the imaging control unit 324.

The operation-history detecting unit 326 detects content of operation, an input of which is received by the operating unit 402 of the endoscope 400, and outputs a result of the detection to the recording unit 34d. Specifically, when an enlarging switch is operated from the operating unit 402 of the endoscope 400, the operation-history detecting unit 326 detects content of the operation and outputs a result of the detection to the recording unit 34d. Note that the operation-history detecting unit 326 may detect operation content of a treatment instrument inserted into the inside of the subject U4 through the endoscope 400 and output a result of the detection to the recording unit 34d.

The recording unit 34d is configured using a volatile memory, a nonvolatile memory, a recording medium, and the like. The recording unit 34d includes an operation-history recording unit 347 instead of the magnification recording unit 346 in the components of the recording unit 34c according to the fifth embodiment explained above.

The operation-history recording unit 347 records a history of operation on the operating unit 402 of the endoscope 400 input from the operation-history detecting unit 326.

The setting unit 38d allocates, based on an attention degree analyzed by the analyzing unit 11 at every predetermined time interval and the operation history recorded by the operation-history recording unit 347, importance and textual information converted by the converter 35 to speech data associated with the same time axis as the time axis of gaze data and records the importance and the textual information in the recording unit 34d. Specifically, the setting unit 38d allocates, based on the attention degree analyzed by the analyzing unit 11 and the operation history recorded by the operation-history recording unit 347, importance (for example, a numerical value) to each frame of the speech data and records the importance in the recording unit 34d. That is, the setting unit 38d performs processing for increasing the importance as a coefficient set according to content of the operation history is larger. The setting unit 38d is configured using a CPU, an FPGA, a GPU, and the like.

The generating unit 39d generates, on an integrated image corresponding to integrated image data generated by the image processing unit 40, gaze mapping data correlated with the attention degree analyzed by the analyzing unit 11 and the textual information and outputs the generated gaze mapping data to the recording unit 34d and the display control unit 323.

The image processing unit 40 combines a plurality of image data recorded by the image-data recording unit 345 to thereby generate integrated image data of a three-dimensional image and outputs the integrated image data to the generating unit 39d.

Processing of the Endoscope System

Processing executed by the endoscope system 300 is explained. FIG. 26 is a flowchart indicating an overview of processing executed by the endoscope system according to the sixth embodiment.

As illustrated in FIG. 26, first, the control unit 32d records each of gaze data generated by the gaze detecting unit 510, speech data generated by the speech input unit 520, and an operation history detected by the operation-history detecting unit 326 in the gaze-data recording unit 341, the speech-data recording unit 342, and the operation-history recording unit 347 in association with a time measured by the time measuring unit 33 (step S601). After step S601, the endoscope system 300 shifts to step S602 explained below.

Steps S602 to S604 respectively correspond to steps S403 to S405 in FIG. 18 explained above. After step S604, the endoscope system 300 shifts to step S605.

In step S605, the setting unit 38d allocates, based on an attention degree analyzed by the analyzing unit 11 at every predetermined time interval and an operation history recorded by the operation-history recording unit 347, importance and textual information converted by the converter 35 to speech data associated with the same time axis as the time axis of the gaze data and records the importance and the textual information in the recording unit 34d.

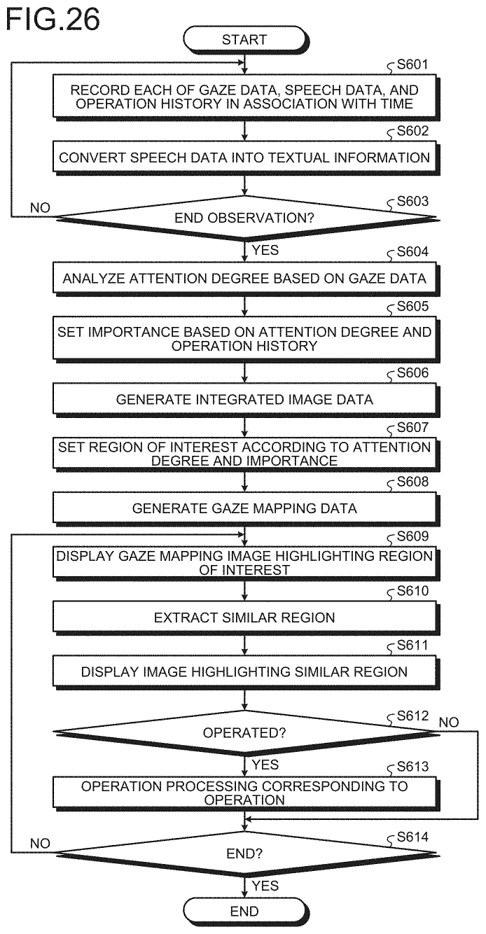

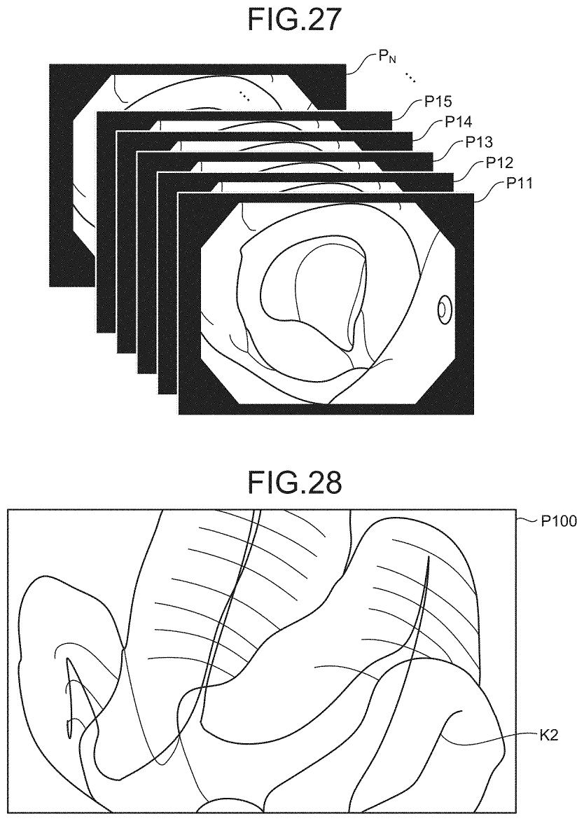

Subsequently, the image processing unit 40 combines a plurality of image data recorded by the image-data recording unit 345 to thereby generate integrated image data of a three-dimensional image and outputs the integrated image data to the generating unit 39d (step S606). FIG. 27 is a diagram schematically illustrating an example of a plurality of images corresponding to the plurality of image data recorded by the image-data recording unit 345. FIG. 28 is a diagram illustrating an example of an integrated image corresponding to the integrated image data generated by the image processing unit. As illustrated in FIGS. 27 and 28, the image processing unit 40 combines a temporally continuous plurality of image data P11 to P_{N} (N=integer) to thereby generate an integrated image P100 corresponding to the integrated image data.

Thereafter, the region-of-interest setting unit 323a sets a region of interest in the integrated image data according to the attention degree analyzed by the analyzing unit 11 and the importance set by the setting unit 38d (step S607).

Subsequently, the generating unit 39d generates, on the integrated image P100 corresponding to the integrated image data generated by the image processing unit 40, gaze mapping data correlated with the attention degree analyzed by the analyzing unit 11, the gaze, the textual information, and the region of interest and outputs the generated gaze mapping data to the recording unit 34d and the display control unit 323 (step S608). In this case, the generating unit 39d may correlate an operation history on the integrated image P100 corresponding to the integrated image data generated by the image processing unit 40 in addition to the attention degree analyzed by the analyzing unit 11, a gaze K2, the textual information, and the region of interest. After step S608, the endoscope system 300 shifts to step S609 explained below.

In step S609, the display control unit 323 superimposes, on an image corresponding to image data, the gaze mapping data highlighting the region of interest and outputs the gaze mapping data to the display unit 20 on the outside. Specifically, the display control unit 323 causes the display unit 20 to highlight and display the region of interest in images of image data P11 to P_{N}.

Subsequently, the similar-region extracting unit 323b extracts a similar region similar to the region of interest in an observation image (step S610). Specifically, the similar-region extracting unit 323b extracts, as the similar region, a region having feature data similar to feature data of the region of interest in the images of the image data P11 to P_{N}.

Thereafter, the display control unit 323 outputs, to the display unit 20 on the outside, an image highlighting the similar region extracted by the similar-region extracting unit 323b on the images of the image data P11 to P_{N} (step S611).

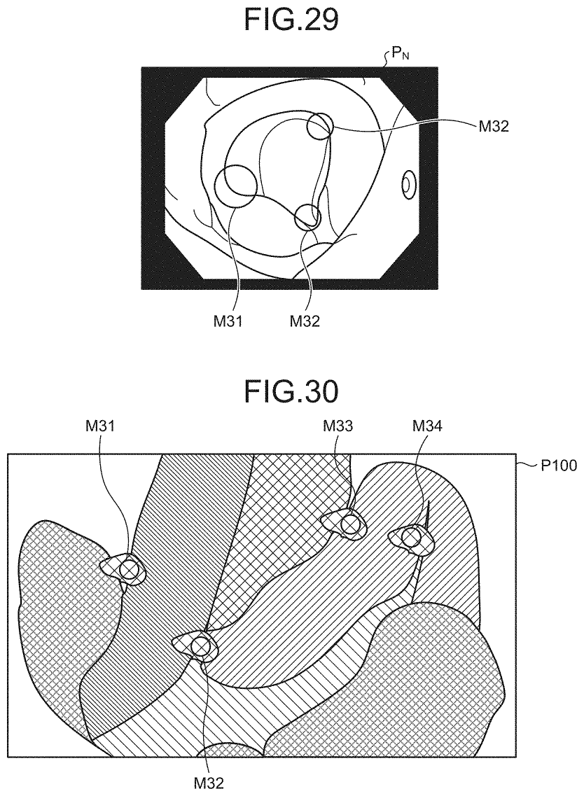

FIG. 29 is a diagram schematically illustrating an example of an image displayed by the display unit according to the sixth embodiment. As illustrated in FIG. 29, the display control unit 323 causes the display unit 20 to display an image highlighting a region of interest M31 and similar regions M32 and M33, for example, in the image data P_{N}. Further, the display control unit 323 may further cause the display unit 20 to display an image highlighting a region of interest and a similar region in the integrated image P100 illustrated in FIG. 28. FIG. 30 is a diagram illustrating a state in which the similar regions are highlighted in FIG. 28. As illustrated in FIG. 30, the display control unit 323 causes the display unit 20 to display an image highlighting the region of interest M31 and the similar regions M32 to M34, for example, in the integrated image P100.

Steps S612 to S614 respectively correspond to steps S412 go S414 in FIG. 18 explained above.

According to the sixth embodiment explained above, the region-of-interest setting unit 323a sets, based on the the attention degree by the gaze and the utterance of the user, the region of interest, which is the region paid attention by the user. The similar-region extracting unit 323b extracts the similar region similar to the region of interest. Consequently, in observation using the endoscope system, it is possible to extract a region similar to a lesion or the like that the user desires to search. As a result, it is possible to efficiently perform diagnosis and prevent a lesion from being overlooked.

Note that, in the sixth embodiment, the similar region is highlighted in the image data P11 to P_{N} and the integrated image P100. However, the similar region may be highlighted in at least one of the image data P11 to P_{N} or the integrated image P100.

In the sixth embodiment, the present disclosure is applied to the endoscope system. However, the present disclosure can also be applied to, for example, an endoscope of a capsule type, a video microscope that captures an image of a subject, a cellular phone having an imaging function, and a tablet terminal having an imaging function.

In the sixth embodiment, the present disclosure is applied to the endoscope system including a flexible endoscope. However, the present disclosure can also be applied to an endoscope system including a rigid endoscope and an endoscope system including an industrial endoscope.

In the sixth embodiment, the present disclosure is applied to the endoscope system including the endoscope inserted into the subject. However, the present disclosure can also be applied to an endoscope system such as a paranasal sinus endoscope, an electric knife, and a test probe.

### OTHER EMBODIMENTS

Various embodiments can be formed by combining, as appropriate, a plurality of components disclosed in the first to sixth embodiments explained above. For example, several components may be deleted from all the components described in the first to sixth embodiments explained above. Further, the components explained in the first to sixth embodiments explained above may be combined as appropriate.

In the first to sixth embodiments, “unit” described above can read “means”, “circuit”, and the like. For example, the control unit can read control means and a control circuit.

A program to be executed by the information processing devices according to the first to sixth embodiments is provided while being recorded in a computer-readable recording medium such as a CD-ROM, a flexible disk (FD), a CD-R, a Digital Versatile Disk (DVD), a USB medium, or a flash memory as file data of an installable form or an executable form.

The program to be executed by the information processing devices according to the first to sixth embodiments may be provided by being stored on a computer connected to a network such as the Internet and downloaded through the network. Further, the program to be executed by the information processing devices according to the first to sixth embodiments may be provided or distributed through a network such as the Internet.

In the first to sixth embodiments, signals are transmitted from the various devices through a transmission cable. However, for example, the signals do not need to be transmitted by wire and may be wirelessly transmitted. In this case, the signals only have to be transmitted from the devices according to a predetermined wireless communication standard (for example, Wi-Fi (registered trademark) or Bluetooth (registered trademark)). Naturally, the wireless communication may be performed according to other wireless communication standards.

Note that, in the explanation of the flowcharts in this specification, an anteroposterior relation of the processing among the steps is clearly indicated using expressions such as “first”, “thereafter”, and “subsequently”. However, the order of the processing necessary for carrying out the disclosure is not uniquely decided by the expressions. That is, the order of the processing in the flowcharts described in this specification can be changed in a range without contradiction.

According to the disclosure, it is possible to realize an information processing device, an information processing method, and a program with which a user can accurately discriminate, in a handsfree manner, a region that the user desires to search in an image.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An information processing device comprising:
a processor comprising hardware, the processor being configured to
analyze, based on gaze data obtained by detecting a gaze of a user and input externally, an attention degree of the gaze of the user with respect to an observation image,
allocate importance corresponding to the attention degree to speech data representing speech of the user, the speech data being input externally and being associated with a same time axis as a time axis of the gaze data,
record the speech data and the importance in a storage, and
set a region of interest in the observation image according to the attention degree and the importance.

2. The information processing device according to claim 1, wherein the processor is further configured to allocate the importance according to the attention degree and an important word that is included in the speech data.

3. The information processing device according to claim 1, the processor is further configured to extract a region similar to the region of interest in the observation image.

4. The information processing device according to claim 1, the processor is further configured to extract a region similar to the region of interest in an image group including images stored in a database.

5. The information processing device according to claim 1, further comprising:
a gaze detector configured to continuously detect gaze of the user to generate the gaze data; and
a speech receiver configured to receive an input of speech of the user to generate the speech data.

6. The information processing device according to claim 5, further comprising:
a microscope including an eyepiece through which the user observes an observation image of a specimen, the microscope being configured to change observation magnification for observing the specimen; and
an imager that is connected to the microscope, the imager being configured to capture the observation image of the specimen formed by the microscope to generate image data, wherein
the gaze detector is provided in the eyepiece of the microscope, and
the processor is further configured to set the region of interest according to the observation magnification.

7. The information processing device according to claim 1, further comprising
an endoscope including:
an imager that is provided at a distal end portion of an insertion portion to be inserted into a subject, the imager being configured to capture an image of an inside of the subject to generate image data; and
an operation receiver configured to receive inputs of various kinds of operation for changing a field of view.

8. An information processing method executed by an information processing device, the information processing method comprising:
analyzing, based on gaze data obtained by detecting a gaze of a user and input externally, an attention degree of the gaze of the user with respect to an observation image;
allocating importance corresponding to the attention degree to speech data representing speech of the user, the speech data being input externally and being associated with a same time axis as a time axis of the gaze data;
recording the speech data and the importance in a storage; and
setting a region of interest in the observation image according to the attention degree and the importance.

9. A non-transitory computer-readable recording medium with an executable program stored thereon, the program causing an information processing device to:
analyze, based on gaze data obtained by detecting a gaze of a user and input externally, an attention degree of the gaze of the user with respect to an observation image;
allocate importance corresponding to the attention degree to speech data representing speech of the user, the speech data being input externally and associated with a same time axis as a time axis of the gaze data;
record the speech data and the importance in a storage; and
set a region of interest in the observation image according to the attention degree and the importance.
